(19) United States
(12) Patent Application Publication
(10) Pub. No.: US 2010/0136137 A1
(43) Pub. Date: Jun. 3, 2010

(54) THERAPEUTIC COMBINATION OF A PANHER/VEGFR2 KINASE INHIBITOR AND A PLATINUM COMPOUND

(75) Inventors: Tai W. Wong, Belle Mead, NJ (US); Jean Soria, Igny (FR); Guido Kroemer, Antony (FR)

Correspondence Address: LOUIS J. WILLE;BRISTOL-MYERS SQUIBB COMPANY, PATENT DEPARTMENT, P O BOX 4000, PRINCETON, NJ, 08543-4000, US

(73) Assignee: Bristol-Meyers Squibb Company

(21) Appl. No.: 12/447,754

(22) PCT Filed: Oct. 31, 2007
(86) PCT No.: PCT/US07/83095

§ 371 Date: Feb. 4, 2010

Related U.S. Application Data
(60) Provisional application No. 60/863,832, filed on Nov. 1, 2006.

(51) Int. Cl. A61K 31/53; A61K 33/24; A61P 35/00

(52) U.S. Cl. 424/649; 514/243

(57) ABSTRACT

This invention relates to a synergistic therapeutic combination of anti-cancer compounds which comprises a) a panHER/VEGFR2 kinase inhibitor, and b) a platinum compound, and optionally at least one pharmaceutically acceptable carrier for simultaneous, separate or sequential use. The invention also relates to treating certain cancers utilizing the combination of the invention.

### FIELD OF THE INVENTION

This invention relates to a synergistic combination of anti-cancer compounds which comprises a) a panHER/VEGFR2 kinase inhibitor, and b) a platinum compound, and optionally one or more pharmaceutically acceptable carriers for simultaneous, separate or sequential use. Additionally, this invention relates to methods for treating cancer employing the combination of the invention.

### BACKGROUND OF THE INVENTION

Platinum compounds have shown activity in a broad spectrum of human tumors in vitro and in vivo. Platinum compounds are antineoplastic and interfere with the growth of cancer cells and slow their growth and spread in the body. The clinical utility of platinum agents in lung, gynecologic and gastrointestinal cancers has been well documented and platinum agents continue to be evaluated in a variety of cancers. Given preclinical evidence of synergy among some platinum compounds and new anticancer agents, clinical trials exploring platinum-based combination therapies may yield improved treatment for a variety of malignancies including lung, breast and genitourinary cancers and myeloma.

Platinum compounds such as cisplatin, carboplatin and oxaliplatin are currently approved for the treatment of various cancers in the U.S. and worldwide.

The panHER/VEGFR2 inhibitor, hereafter called “Compound I”, has been found to target two crucial signaling pathways, namely HER-mediated signaling and angiogenesis. As such, it is currently being evaluated in clinical trials for the treatment of various cancers including lung cancer.

The nature of proliferative diseases like solid tumor diseases is multifactorial. Under certain circumstances, drugs with different mechanisms of action may be combined. However, just considering any combination of drugs having different modes of action does not necessarily lead to combinations with advantageous effects. In fact, drugs within the same class may not all have the same effect when used in combination.

### SUMMARY OF THE INVENTION

This invention relates to a synergistic combination of anti-cancer compounds which comprises a) a panHER/VEGFR2 kinase inhibitor, and b) a platinum compound, and optionally one or more pharmaceutically acceptable carriers for simultaneous, separate or sequential use.

In particular, it has been found that Compound I of the formula

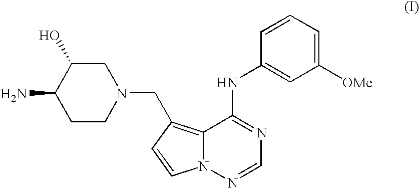

when administered following a platinum compound, exhibited therapeutically synergistic antitumor activity in certain non-small cell lung cancer (NSCLC) cell lines.

More particularly, it has been found that Compound I, when administered following the platinum compound, cisplatin, exhibited therapeutically synergistic cell growth inhibition in certain NSCLC cell lines.

### BRIEF DESCRIPTION OF THE DRAWINGS

FIG. 1. Synergistic combined therapy using Compound I, plus the platinum compound, cisplatin, in A549 cells. Cell proliferation is shown in the legend.

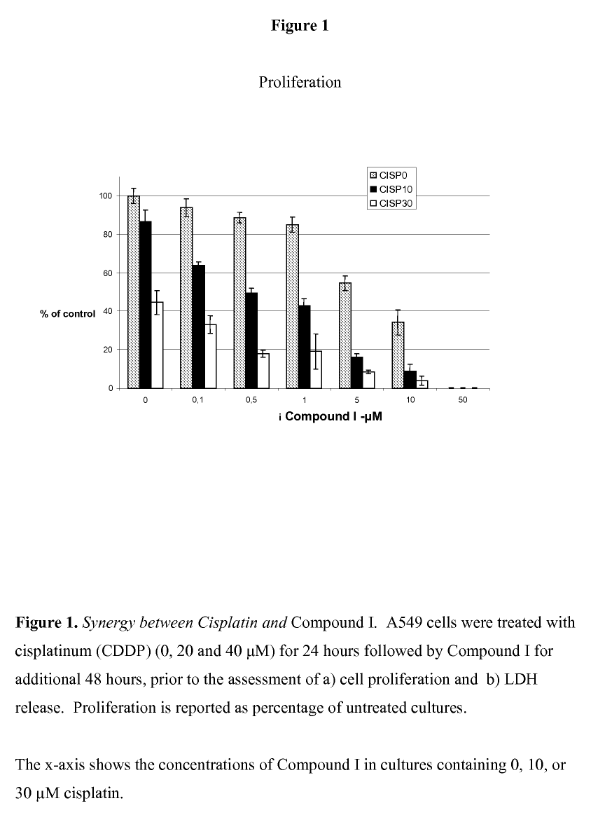

FIG. 2. Synergistic combined therapy using Compound I, plus the platinum compound, cisplatin, in A549 cells. LDH release is shown in the legend.

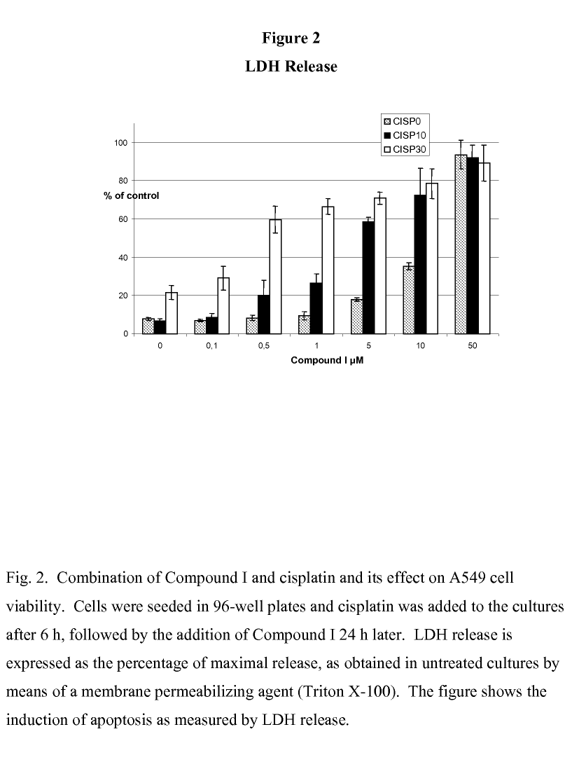

### DETAILED DESCRIPTION OF THE INVENTION

It has been found that certain panHER/VEGFR2 kinase inhibitors, when administered sequentially with a platinum compound, exhibit therapeutically synergistic cell growth inhibition in certain NSCLC cell lines. This showing could correlate to their use for the treatment of certain types of cancers. The invention also relates to methods of treating cancer and other proliferative diseases using the synergistic therapeutic combination of compounds.

Compound I, 4-Amino-1-[[4-[(3-methoxyphenyl)amino]pyrrolo [2,1-f][1,2,4]triazin-5-yl]methyl]-(3R,4R)-3-piperidinol, having the following structure

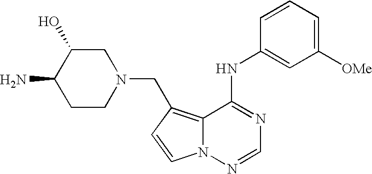

or a pharmaceutically acceptable salt or stereoisomer thereof, is used in the combination and methods of the invention. This compound and its preparation is disclosed in U.S. Ser. No. 11/019,901, filed Dec. 22, 2004, the disclosure of which is incorporated herein by reference.

Additional panHER/VEGFR2 kinase inhibiting compounds such as those disclosed in U.S. Ser. No. 11/019,901, filed Dec. 22, 2004, may also be useful in the synergistic combination of the invention.

The platinum compound can be selected from cisplatin, carboplatin and oxaliplatin. These compounds have been approved by the FDA for the treatment of cancer either as monotherapy or for the adjuvant treatment of cancers.

The platinum compound, cisplatin, was found to provide the therapeutically synergistic cell inhibition activity when combined with Compound I.

It has been surprisingly found that the combination of a dosage unit of the panHER/VEGFR2 kinase inhibitor of formula I plus a dosage unit of the platinum compound, cisplatin, provided therapeutically synergistic cell growth inhibition in certain NSCLC cell lines, including cell line A549.

It can be shown by established test models and in particular those models described herein that the combination of the invention results in synergistic activity compared to the effects observed with the compounds administered alone. The pharmacological activity of the combination of the invention may be further demonstrated in a clinical study as well as in the procedure described herein.

Therapeutic synergy represents a therapeutic effect achieved with a tolerated regimen of a combination treatment that exceeds the optimal effect achieved at any tolerated dose of monotherapy associated with the same drugs used in the combination.

The following are definitions of terms that may be used in the present specification. The initial definition provided for a group or term herein applies to that group or term throughout the present specification individually or as part of another group, unless otherwise indicated.

The term “platinum compound’ as used herein means carboplatin, cisplatin or oxaliplatin.

The term “carboplatin” as used herein relates to the antineoplastic agent cis-daimine (1,1-cyclobutane dicarboxylato)platinum (II), which is disclosed, e.g., in U.S. Pat. No. 4,140,707 and U.S. Pat. No. 4,657,927. This drug can be administered, for example, in the form as it is marketed, e.g., under the trademark CARBOPLAT® or PARAPLATIN®.

The term “oxaliplatin” as used herein refers to the antineoplastic agent also known as oxalatoplatinum, which is disclosed, e.g., in U.S. Pat. No. 5,716,988. This drug can be administered, for example, in the form as it is marketed, e.g., under the trademark ELOXATIN®.

The term “cisplatin” as used herein refers to the antineoplastic agent also known as diaminedichloroplatinum, a pharmaceutical composition of which is disclosed, e.g., in U.S. Pat. No. 4,310,515. This drug can be administered, for example, in the form as it is marketed, e.g., under the trademark PLATINOL® or PLATINOL-AQ®.

The term “dosage unit” as used herein means a therapeutic amount or sub-therapeutic amount of the compound utilized in the combination of the invention. For approved drugs, this amount would be shown in the PDR and for compounds in clinical trials; this amount would be based on the ongoing trials and the skill of one in the art.

When a compound according to this invention is administered into a human subject, the daily dosage will normally be determined by the prescribing physician with the dosage generally varying according to the age, weight, sex and response of the individual patient, as well as the severity of the patient's symptoms.

Compound I may form salts which are also within the scope of this invention. Pharmaceutically acceptable (i.e. non-toxic, physiologically acceptable) salts are preferred, although other salts are also useful, e.g., in isolating or purifying the compounds of this invention.

The compounds of formula I may form salts with alkali metals such as sodium, potassium and lithium, with alkaline earth metals such as calcium and magnesium, with organic bases such as dicyclohexylamine, tributylamine, pyridine and amino acids such as arginine, lysine and the like. Such salts can be formed as known to those skilled in the art.

The compounds for formula I may form salts with a variety of organic and inorganic acids. Such salts include those formed with hydrogen chloride, hydrogen bromide, methanesulfonic acid, sulfuric acid, acetic acid, trifluoroacetic acid, oxalic acid, maleic acid, benzenesulfonic acid, toluenesulfonic acid and various others (e.g., nitrates, phosphates, borates, tartrates, citrates, succinates, benzoates, ascorbates, salicylates and the like). Such salts can be formed as known to those skilled in the art.

In addition, zwitterions (“inner salts”) may be formed.

All stereoisomers of the compounds of the instant invention are contemplated, either in admixture or in pure or substantially pure form. The definition of compounds according to the invention includes all the possible stereoisomers and their mixtures. Particularly preferred are the racemic forms and the isolated optical isomers having the specified activity. The racemic forms can be resolved by physical methods, such as, for example, fractional crystallization, separation or crystallization of diastereomeric derivatives or separation by chiral column chromatography. The individual optical isomers can be obtained from the racemates from the conventional methods, such as, for example, salt formation with an optically active acid followed by crystallization.

The combination of the invention may be useful in the treatment of a variety of cancers, including (but not limited to) the following:
carcinoma, including that of the bladder, breast, colon, kidney, liver, lung, including small cell lung cancer and non-small cell lung cancer, esophagus, gall bladder, ovary, pancreas, stomach, cervix, thyroid, prostate, and skin, including squamous cell carcinoma;
hematopoietic tumors of lymphoid lineage, including leukemia, acute lymphocytic leukemia, acute lymphoblastic leukemia, B-cell lymphoma, T-cell lymphoma, Hodgkins lymphoma, non-Hodgkins lymphoma, hairy cell lymphoma and Burkitt's lymphoma;
hematopoietic tumors of myeloid lineage, including acute and chronic myelogenous leukemias, myelodysplastic syndrome and promyelocytic leukemia;
tumors of mesenchymal origin, including fibrosarcoma and rhabdomyosarcoma;
tumors of the central and peripheral nervous system, including astrocytoma, neuroblastoma, glioma and schwannomas; and
other tumors, including melanoma, seminoma, teratocarcinoma, osteosarcoma, xenoderoma pigmentosum, keratoctanthoma, thyroid follicular

One benefit is that lower doses of the active ingredients of the combination of the invention may be used. Additionally, the dosages need not only be smaller but administered less frequently or used to diminish the incidence of side effects.

The effective dosage of each of the combination partners employed may vary depending on the particular compound or pharmaceutical composition employed. Thus, the dosage regimen is selected in accordance with a variety of factors including the route of administration and the renal and hepatic function of the patient. A physician or clinician of ordinary skill can readily determine and prescribe the effective amount of the single active ingredients required to prevent, counter or arrest the progress of the condition. Optimal precision in achieving concentration of the active ingredients within the range that yields efficacy without toxicity requires a regimen based on the kinetics of the active ingredients' availability to target sites. This involves a consideration of the distribution, equilibrium and elimination of the active ingredients.

### EXAMPLES

### Materials and Methods

Compounds. Compound I was synthesized by Bristol-Myers Squibb (BMS) chemists.

Non-Small Cell Lung Cancer cell lines with different EGFR and p53 mutation status were used. Variable doses of Compound I or cisplatin were used to induce death or proliferative death of the cells. A549 cells were transfected with siRNAs, and transfected cells were cultured for 48 hours prior to the administration of the drugs to attain the maximal down-regulation of the target proteins.

The fluorochromes, 3,3′-dihexyloxacarbocyanine iodide (DiOC63) and propidium iodide (PI) were employed to determine different apoptosis-associated changes. DiOC63 was used for the loss of mitochondrial transmembrane potential (Δψₘ) and PI was used for the loss of viability.

### Results

Compound I induced anti-proliferative/pro-apoptotic effects in all NSCLC cell lines tested (including those carrying wild-type EGFR and those which encode the T790M mutant). The following order of sensitivity was found: NCI-H1975>>NCI-H1650=NCIH1299>A549. Compound I induced dissipation of Δψₘ and incorporation of PI, two markers associated with early and late apoptosis, respectively. Interestingly, the pan-caspase inhibitor, z-VAD.fmk had only a minor protective effect on the reduction of the Δψₘ and had no effect on the loss of viability.

The combined treatment of A549 cells with Compound I and cisplatin resulted in synergy in growth inhibition, while either drug administered alone had only minor growth-inhibitory effects. In particular, 500 nM to 1 μm of Compound I synergized with 10 μm of cisplatin to induce 50-60% of cell growth inhibition while either compound administered alone had only minor growth inhibitory effect (10-15%). Synergy occurred only when Compound I was administered 24 hours later than cisplatin and not when the drugs were added in the reverse order. SiRNA knockdown was used to determine the role of components of apoptosis pathways and DNA repair in the mechanism of action of Compound I. Down-regulation of caspase-2 provided limited protection against Compound I induced cell-death at 24 hours, but not at 48 hours. Bcl-2 down-regulation, alone or I combination with Bcl-X_{L} strongly sensitized A549 cells to the effect of Compound I, at both 24 and 48 hours.

### CONCLUSION

Compound I was able to arrest proliferation and to induce apoptosis at low doses in all tested NSCLC cell lines, including cells harboring the EGFR T790M mutation (T790M). The pro-apoptotic effects of Compound I involves caspase-dependent as well as caspase-independent mechanisms in cell killing. Compound I sensitized NSCLC cells to the anti-proliferative and pro-apoptotic activity of cisplatin, in a sequence-dependent manner, suggesting that the cisplatin-induced cell cycle arrest may render cells more sensitive to the effects of Compound I.

In conclusion, Compound I may become a viable alternative to current EGFR-TKIs (tyrosine kinase inhibitors), like erlotinib and gefitinib, particularly in an optimized combination regimen.

We claim:

1. A synergistic pharmaceutical combination of anti-cancer compounds which comprises administering to a mammal a dosage unit of
a) a panHER/VEGFR2 kinase inhibitor, and a dosage unit of
b) a platinum compound,
in which the active ingredients are present in each case in free form or as a pharmaceutically acceptable salt or stereoisomer thereof.

2. The combination according to claim 1 wherein the panHER/VEGFR2 kinase inhibitor is Compound I of the formula 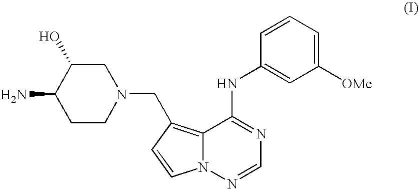
or a pharmaceutically acceptable salt or stereoisomer thereof.

3. The combination according to claim 1 wherein the platinum compound is selected from the group consisting of cisplatin, carboplatin and oxaliplatin.

4. The combination according to claim 3 wherein the panHER/VEGFR2 kinase inhibitor is Compound I of the formula 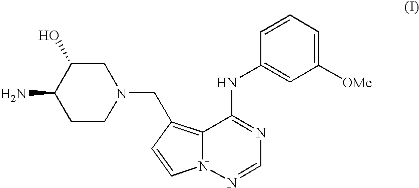
or a pharmaceutically acceptable salt or stereoisomer thereof, and the platinum compound is selected from the group consisting of cisplatin, carboplatin and oxaliplatin.

5. The combination according to claim 4 wherein the platinum compound is cisplatin.

6. The combination according to claim 3 wherein the panHER/VEGFR2 kinase inhibitor is Compound (I) or a pharmaceutically acceptable salt or stereoisomer thereof, and the platinum compound is cisplatin.

7. The combination according to claim 6 wherein the two compounds are administered sequentially.

8. The combination according to claim 7 wherein Compound I is administered following the administration of the platinum compound.

9. A method for treating cancer which comprises administering to a mammal a combination therapy comprising a dosage unit of
a) a panHER/VEGFR2 kinase inhibitor, and a dosage unit of
b) a platinum compound,
in which the active ingredients are present in each case in free form or as a pharmaceutically acceptable salt or stereoisomer thereof,
wherein the combination therapy will provide a greater anti-cancer effect than the effect obtainable with either the dosage unit of Compound (1) or the platinum compound alone.

10. The method of claim 9 wherein the panHER/VEGFR2 kinase inhibitor is a compound of the formula 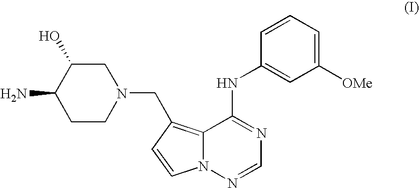
or a pharmaceutically acceptable salt or stereoisomer thereof, and the platinum compound is selected from the group consisting of cisplatin, carboplatin and oxaliplatin.

11. The method of claim 10 wherein the wherein the panHER/VEGFR2 kinase inhibitor is a compound of formula (I) or a pharmaceutically acceptable salt or stereoisomer thereof, and the platinum compound is cisplatin.

12. The method according to claim 11 wherein the two compounds are administered sequentially.

13. The method according to claim 12 wherein Compound I is administered following the administration of the platinum compound.

14. The method according to claim 9 wherein the cancer treated is selected from colorectal cancer, breast cancer, gastric cancer, ovarian cancer, non-small cell lung cancer and cancers of the head and neck.

15. The method according to claim 14 wherein the cancer is non-small cell lung cancer.
